(19) United States
(12) Patent Application Publication
(10) Pub. No.: US 2024/0408156 A1
(43) Pub. Date: Dec. 12, 2024

(54) BACTERIOPHAGE PREPARATIONS AND METHODS OF USE

(71) Applicants: UNM Rainforest Innovations, Albuquerque, NM (US); The Regents of the University of California, Oakland, CA (US); The United States Government As Represented By The Department of Veterans Affairs, Washington, DC (US)

(72) Inventors: Henry C. Lin, Albuquerque, NM (US); Derek M. Lin, Albuquerque, NM (US); Britt Koskella, Berkeley, CA (US)

(21) Appl. No.: 18/700,605

(22) PCT Filed: Oct. 12, 2022
(86) PCT No.: PCT/US2022/046449

§ 371 Date: Apr. 11, 2024

Related U.S. Application Data
(60) Provisional application No. 63/255,105, filed on Oct. 13, 2021.

(51) Int. Cl. A61K 35/76; A61K 9/00; A61P 1/00; C12N 7/00

(52) U.S. Cl. CPC A61K 35/76; A61K 9/0053; A61P 1/00; C12N 7/00; C12N 2795/00032

(57) ABSTRACT

A bacteriophage preparation generally includes a preparation of a heterogeneous bacteriophages obtained from a fermentative process. The bacteriophage preparation may be formulated into a pharmaceutic composition. The bacteriophage preparation may be used to treat dysbiosis by administering the bacteriophage preparation to a subject having, or at risk of having, dysbiosis. The bacteriophage preparation may be used to prepare or prime the gut environment of a healthy subject prior to administering a source of bacteria to the subject.

### CROSS-REFERENCE TO RELATED APPLICATION

This application claims the benefit of U.S. Provisional Patent Application No. 63/255,105, filed Oct. 13, 2021, which is incorporated herein by reference in its entirety.

### SUMMARY

This disclosure describes, in one aspect, a pharmaceutical composition that includes a preparation of a heterogeneous bacteriophages obtained from a fermentative process and a pharmaceutically-acceptable carrier. In one or more embodiments, the pharmaceutical composition of can further include an adjuvant.

In one or more embodiments, the pharmaceutical composition includes bacteriophages that are members of the family Myoviridae, members of the family Podoviridae, members of the family Siphoviridae, members of the family Inoviridae, members of the family Microviridae, members of the family members of the family Corticoviridae, members of the family Tectiviridae, members of the family Leviviridae, members of the family Cystoviridae, members of the family Rudiviridae, members of the family Fuselloviridae, members of the family Lipothrixviridae, members of the family Plasmaviridae, or bacteriophages from a combination of the foregoing families.

In one or more embodiments, the fermentative process produces a food. In other embodiments, the fermentative process produces a fertilizer or livestock feed.

In one or more embodiments, the fermentative process includes fermentation of a plant material, a plant juice, a feedstock, a biofuel, or a bio-waste.

In one or more embodiments, the pharmaceutical composition is formulated for delivery to epithelial tissue.

In another aspect, this disclosure describes a method of preparing a bacteriophage preparation. Generally, the method includes isolating bacteriophage from a fermentative process and combining the isolated bacteriophage with a pharmaceutically acceptable carrier.

In one or more embodiments, the method further includes enriching the bacteriophage preparation so that the concentration of bacteriophages in the preparation is increased, the bioactivity of bacteriophages in the preparation is increases, or both.

In one or more embodiments, the method further includes culturing the bacteriophage preparation with suitable bacterial species in a bioreactor.

In one or more embodiments, the fermentative process produces a food. In other embodiments, the fermentative process produces a fertilizer or livestock feed.

In one or more embodiments, the fermentative process includes fermentation of a plant material, a plant juice, a feedstock, a biofuel, or a bio-waste.

In another aspect, this disclosure describes a method of treating dysbiosis in a subject having, or at risk of having, dysbiosis. Generally, the method includes administering to the subject a bacteriophage preparation in an amount effective to ameliorate at least one symptom or clinical sign of dysbiosis.

In one or more embodiments, the method further includes administering to the subject a second pharmaceutical composition for treating dysbiosis. In some of these embodiments, the second pharmaceutical composition for treating dysbiosis can include an antibiotic, a prebiotic, a probiotic, a synbiotic agent, a fecal microbiota transplant, membrane vesicles, or an autophagy inducer.

In one or more embodiments, the dysbiosis is localized to an epithelial tissue. In some of these embodiments, the epithelial tissue includes epidermis, a mucosal surface, at least a portion of the skin or scalp, at least a portion of the oral cavity, at least a portion of the gastrointestinal tract, at least a portion of the nasal cavity, at least a portion of the respiratory tract, at least a portion of the genito-urinary tract, or a body cavity.

In one or more embodiments, the bacteriophage preparation is incorporated into a medical device.

In one or more embodiments, the dysbiosis causes bacterial overgrowth.

In one or more embodiments, the method further includes subjecting the subject to a sustained period of nutrient deprivation prior to administering the bacteriophage preparation to the subject. In some of these embodiments, the sustained period of nutrient deprivation is about one to three days.

In one or more embodiments, the method further includes subjecting the subject to one or more meals prior to, concurrently with or after administering the bacteriophage preparation to the subject.

In one or more embodiments, the bacteriophage preparation is administered orally, administered topically, administered through inhalation, or administered parenterally.

In yet another aspect, this disclosure describes a method of preparing or priming the gut environment of a healthy subject for administering a source of bacteria. Generally, the method includes administering to the subject a bacteriophage preparation in an amount effective to improve receptivity of subject's gut to the source of bacteria.

In one or more embodiments, the source of bacteria comprises a probiotic, one or more than one (e.g., a cocktail) bacteria grown in culture, or a fecal microbiota transplant.

In one or more embodiments, the bacteriophage preparation is administered by mouth, delivered directly to the gut, or encapsulated as a microbial transplant.

In one or more embodiments, the method further includes subjecting the subject to a sustained period of nutrient deprivation prior to administering the bacteriophage preparation to the subject. In some of these embodiments, the sustained period of nutrient deprivation is about one to three days.

In one or more embodiments, the method further includes subjecting the subject to a sustained period of nutrient deprivation prior to administering the bacteriophage preparation to the subject. In some of these embodiments, the sustained period of nutrient deprivation is about one to three days.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Water avoidance stress (WAS)-tested animals who received a PBS placebo (WAS-PBS) had a 3.3-fold increase in bacterial density compared to control animals (p<0.005), demonstrating psychological stress-induced bacterial overgrowth. However, the bacterial density in WAS-tested mice that received the bacteriophage preparation (WAS-Phage) was not significantly different from the control animals, demonstrating effectiveness of the bacteriophage preparation in inhibiting stress-induced bacterial overgrowth. All results were represented as a fold change of 16S rRNA gene copy number relative to that of control (set at 1.0).

FIG. 2. WAS-tested animals who received either a PBS placebo (WAS-PBS) or a bacteriophage preparation (WAS-Phage) had significantly different beta diversity compared to control animals (p<0.01), as measured by the Bray-Curtis dissimilarity index. These data demonstrate that testing with WAS is associated with changing the overall composition of the gut microbiome.

FIG. 3. Only WAS-tested animals who received a bacteriophage preparation (WAS-Phage) had an increased alpha diversity relative to control (p<0.05), as measured using the Chao1 alpha diversity index, whereas the WAS-tested animals receiving a PBS placebo (WAS-Phage) did not have a significantly increased alpha diversity relative to control (p=0.2). These data on alpha diversity, interpreted with the data from FIG. 1 and FIG. 2, demonstrate the effectiveness of the bacteriophage preparation in increasing overall gut microbiome diversity in the setting of stress-induced gut microbiome dysbiosis.

### DETAILED DESCRIPTION OF ILLUSTRATIVE EMBODIMENTS

This disclosure describes pharmaceutical compositions and methods involving the use of bacteriophages for alleviating dysbiosis.

In the setting of the gut microbiome, dysbiosis is an abnormal state in which the microbiome is associated with numerous health disorders and adverse conditions. A dysbiotic microbiome can typically be characterized by reduced overall diversity of the bacterial membership and increased density of select bacterial members when compared to a healthy microbiome. Similarly, a dysbiotic gut virome can typically be characterized by reduced overall diversity of the viral membership and increased density of select viral members when compared to a healthy gut virome. Conversely, greater bacterial and/or viral diversity is associated with a healthy gut microbiome. Whereas many bacterial infections typically involve a single bacterial pathogen, as in the case of methicillin-resistant Staphylococcus aureus infections, dysbiosis may involve many members or even the entire community of the >500-1000 bacterial species that make up the gut microbiome, many of which are resident, symbiotic, and non-pathogenic. Gut microbiomes interact closely with their human host, and this interaction directly affects human health. Additionally, other relevant components of the gut ecosystem that exhibit lower diversity in the setting of dysbiosis are the archaeome (archaea), phageome (bacteriophages), virome (viruses), fungome (fungi), metagenome (total catalog of genes), metatranscriptome (total catalog of genes expressed), metaproteome (total catalog of proteins).

Dysbiosis is associated with many disorders including, but not limited to, chronic non-communicable diseases of developed society (atopias, metabolic syndrome, inflammatory diseases, cancer, some behavior disorders, etc.), irritable bowel syndrome (IBS, including IBS-related gastrointestinal, neurological, and psychiatric pathologies and IBS-associated neurodegenerative and psychiatric comorbidities), chronic fatigue syndrome, fibromyalgia, gastroesophageal reflux disease, functional abdominal pain and other functional disorders, Crohn's Disease, ulcerative colitis, inflammatory bowel disease (IBD), eating disorders, idiopathic pulmonary fibrosis (IPF), autoimmune disorders, COVID-19, post-COVID-19 long haul syndrome, interstitial cystitis, chronic pelvic pain syndrome, chronic prostatitis, chronic epididymitis, cirrhosis, hyperdynamic circulation, ascites, varices, encephalopathy, renal insufficiency, hepatorenal syndrome, microbial translocation, end stage renal disease, celiac disease, allergies, asthma, gluten sensitivity or gluten intolerance, cardiovascular disease, Gulf War Syndrome or Illness, attention deficit hyperactivity disorder (ADHD), post-traumatic stress disorder, anxiety disorder, diabetes, metabolic syndrome, glucose intolerance, leaky gut syndrome, gut barrier dysfunction, hypersensitivity disorders, cystic fibrosis, multi-symptom disorder, environmental illness, food intolerance and food allergies, food sensitivity, multiple food or chemical sensitivity, autism, post-traumatic depression, neurodegeneration, multiple sclerosis, Alzheimer's disease, mild cognitive impairment, Parkinson's Disease, peripheral neuropathy, dementias with Lewy bodies (DLB), idiopathic REM sleep behavior disorder (iRBD), restless leg syndrome, sleep disorders, obstructive sleep apnea, postural hypotension, postural orthostatic syndrome, systemic lupus erythematosus, scleroderma, rheumatoid arthritis (RA), osteoarthritis (OA), skin disorders (e.g., acne, atopic dermatitis, psoriasis, rosacea, urticaria, acne vulgaris, bullous pemphigus, etc.), Raynaud's syndrome, osteoarthritis, Sjorgren's syndrome and other autoimmune disorders, atherosclerosis and related cardiovascular diseases, costochondritis, hyperhomocysteinemia, Hashimoto's thyroiditis, atopic dermatitis, eczema, vaginosis, gingivitis, periodontal disease, disease of pulp chamber of a tooth, dental caries, uveitis, iritis, hyperalgesia, opioid-induced hyperalgesia, allodynia, opioid bowel syndrome, narcotic hypersensitivity syndrome, hematopoietic-cell transplantation, disorders of protective antibacterial mechanisms (e.g., achlorhydria, pancreatic exocrine insufficiency, an immunodeficiency syndrome, etc.), anatomical abnormalities (e.g., small intestinal obstruction, diverticula, fistulae, surgical blind loop, previous ileo-caecal resection, etc.) motility disorders (e.g., scleroderma, autonomic neuropathy in diabetes mellitus, post-radiation enteropathy, small intestinal pseudo-obstruction, etc.), mastitis, malnutrition, critical illnesses, chronic diseases, oral mucositis, intestinal mucositis, Candida albicans infection, diabetes mellitus, atopic diathesis, allergic rhinoconjunctivitis, food allergy, eosinophilic esophagitis, pancreatitis, butyrate depletion, infertility, non-alcoholic steatohepatitis (NASH), non-alcoholic fatty liver disease (NAFLD), alcoholic liver disease (ALD), liver cancer, liver disease, liver transplantation, bronchiectasis, chronic obstructive pulmonary disease (COPD), gastrointestinal carcinomas, esophageal carcinoma, gastric carcinoma, colorectal carcinoma, pancreatic carcinoma, hepatocellular carcinoma, surgical-site infection, enteritis, colorectal cancer, hepatic steatosis, graft-versus-host disease (GVHD), oral squamous cell carcinoma, sarcopenia, urinary stone disease, kidney disease, Clostridium difficile infection (CDI), allogenic stem cell transplantation, osteopathogenic disorders, fungal translocation and facilitate invasive disease, fungal bloodstream infection (e.g., Candida bloodstream infection, etc.), chronic rhinosinusitis (CRS), neuroinflammation, autism spectrum disorder, attenuated morphine analgesic intolerance, retinal degenerative diseases (e.g., retinitis pigmentosa, etc.), necrotizing enterocolitis (NEC), hepatic encephalopathy, cirrhosis-related cognitive performance, addiction disorders, aging, chronic otitis media, immunosenescence, urgency urinary incontinence, chronic kidney disease, vitiligo, insulin resistance, vaginal microbiome dysbiosis, nasopharyngeal carcinoma, hypertension, dyslipidemia, idiopathic intracranial hypertension syndrome (IIH), peri-implant mucositis (PM), peri-implantitis (PI), cardiometabolic health during menopause, senile pruritus (SP), primary biliary cholangitis, sensitive scalp, seborrheic dermatitis, autoimmune thyroid diseases, Graves-Basedow's disease, bacterial vaginosis, sexually transmitted infections, glaucoma, recurrent miscarriage, recurrent implantation failure, atherogenic dyslipidemia, vertical sleeve gastrectomy, axial sponyloarthritis, chronic psychological stress, age-associated declines in microbiota diversity, urinary tract infection, visceral hypersensitivity, intestinal membrane permeability, fibromyalgia, schizophrenia, attention deficit/hyperactivity disorder, leaky gut syndrome, polycystic ovary syndrome, ischemic stroke, atherosclerotic plaque development, atherothrombosis, microscopic colitis, and substance use disorder.

Dysbiosis also may be associated with chemotherapy, hemodialysis, antibiotic use (e.g., in infancy), bariatric surgery, and being placed on mechanical ventilation.

Symptoms and clinical signs of disorders associated with dysbiosis include, but are not limited to fatigue, low energy, flu-like illness, poor sleep, insomnia, panic, depression, bone loss, boborygmi, bloating, flatulence, distension, heartburn, dyspepsia, picky eating, constipation, diarrhea (e.g., refractory diarrhea, bile acid diarrhea, antibiotic-associated diarrhea, etc.), altered vagal gut-brain communication, abdominal pain, paresthesia, tingling, neuropathy, fogginess of head, brain fog, halitosis, sugar craving, depression, anxiety, poor psychiatric outcomes, impaired mentation, difficulty with concentration, dementia, mild cognitive impairment, impaired memory, synaptic plasticity, moodiness, irritability, headaches, migraines, dizziness, aches and pains, chronic widespread musculoskeletal pain, shortness of breath, nausea or vomiting, poor appetite, increased appetite, acne, rash, cold hand and feet, involuntary weight loss, or obesity.

One type of dysbiosis that is associated with decreased bacterial density is small intestinal bacterial overgrowth (SIBO), which is present in up to 78% of patients with irritable bowel syndrome. SIBO is characterized by reduced diversity in the gut microbial community and/or excess bacterial colonization in the proximal end of the small intestine, which may be independent of excess bacterial density in the more distal part of the gastrointestinal tract. Intestinal bacterial overgrowth describes excess bacterial density in proximal and/or distal parts of the gastrointestinal tract. In the case of SIBO, this overgrowth is not limited to one or a few bacterial species. Rather, like many dysbioses, SIBO can include many or all species of the gut microbiome and is commonly caused by increased density of resident non-pathogenic bacterial species that become pathobionts. In a healthy gastrointestinal tract, the proximal end of the small intestine is virtually sterile, with gradually increasing bacterial density towards the distal end. Increased colonization of gut bacteria in the small intestine, especially when into the more proximal end of the small intestine, is associated with triggering host immunity, which leads to an increased proinflammatory/immune response, leaky gut, impaired intestinal barrier function, and/or bacterial translocation across the intestinal barrier and into the bloodstream.

Thus, SIBO and intestinal bacterial overgrowth is associated with many disease states, symptoms, and/or clinical signs including, but not limited to irritable bowel syndrome (IBS), chronic fatigue syndrome, fibromyalgia, gastroesophageal reflux disease, functional abdominal pain and other functional disorders, Crohn's Disease, ulcerative colitis and other inflammatory bowel disease, post-COVID-19 long haul syndrome, interstitial cystitis, chronic pelvic pain syndrome, chronic prostatitis, chronic epididymitis, cirrhosis, hyperdynamic circulation, ascites, varices, encephalopathy, renal insufficiency, hepatorenal syndrome, microbial translocation, end stage renal disease, celiac disease, allergies, asthma, gluten sensitivity or gluten intolerance, cardiovascular disease, Gulf War Syndrome or Illness, attention deficit hyperactivity disorder (ADHD), post-traumatic stress disorder, anxiety disorder, diabetes, metabolic syndrome, glucose intolerance, leaky gut syndrome, gut barrier dysfunction, hypersensitivity disorders, cystic fibrosis, multi-symptom disorder, environmental illness, food intolerance and food allergies, food sensitivity, multiple food or chemical sensitivity, autism, post-traumatic depression, multiple sclerosis, Alzheimer's disease, mild cognitive impairment, Parkinson's Disease, peripheral neuropathy, restless leg syndrome, sleep disorders, postural hypotension, postural orthostatic syndrome, systemic lupus erythematosus, multiple sclerosis, scleroderma, rheumatoid arthritis, psoriasis, rosacea, urticaria, Raynaud's syndrome, osteoarthritis, Sjorgren's syndrome and other autoimmune disorders, atherosclerosis and related cardiovascular diseases, costochondritis, hyperhomocysteinemia, Hashimoto's thyroiditis, atopic dermatitis, eczema, vaginosis, gingivitis, periodontal disease, disease of pulp chamber of a tooth, dental caries, uveitis, or iritis, hyperalgesia, opioid-induced hyperalgesia, allodynia, opioid bowel syndrome, narcotic hypersensitivity syndrome.

Available treatments for SIBO and intestinal bacterial overgrowth are limited to antibiotic therapies including, for example, rifaximin, doxycycline, augmentin, ciprofloxacin, metronidazole, norfloxacin, or neomycin, which have varying degrees of clinical effectiveness and therefore are not successful in all patients.

This disclosure describes compositions and methods involving the use of bacteriophages for treating dysbioses exemplified by, but not limited to, SIBO, intestinal bacterial overgrowth, and dysbiotic conditions characterized by a reduction or loss of bacterial diversity.

Bacteriophages are ubiquitous viruses that infect bacterial hosts. Each bacteriophage depends on a bacterial host for reproduction and has a limited range of susceptible bacterial hosts. One of the ways in which a bacteriophage replicates through its bacterial host involves the bacteriophage attaching to a receptor on the outside of a bacterial cell, injecting its bacteriophage DNA into the bacterial chromosome, replication of bacteriophage particles by the bacterial host, and lysis of the bacterial host, which ultimately results in a release of new bacteriophage progeny that find new hosts to repeat the cycle.

In microbial communities such as the gut microbiome, community-wide infection of bacteria by bacteriophages is a mechanism that helps regulate bacterial community composition by downregulating the abundance of bacterial species with a high population density, a process that helps drive and maintain diversity of bacterial species. Bacteriophages are abundant in the gut microbiome, amounting to approximately 10¹³ bacteriophages in the gut, a population approximately the same size as gut bacteria. Bacteriophage-bacteria interactions are foundational to healthy microbial communities, including a healthy gut microbiome, and dysfunction in bacteriophage-bacteria dynamics is associated with reduced bacterial diversity and increased density of specific bacterial members (i.e., dysbiosis).

The coevolutionary relationship between bacteriophages and bacteria, in which bacteriophages regulate the growth and density of bacterial species, coupled with the ability of bacteriophages to replicate and multiply, gives bacteriophage-based gut microbiome interventions the potential to address SIBO, intestinal bacterial overgrowth, and dysbiotic conditions characterized by a reduction or loss of bacterial diversity, and other forms of dysbioses. Moreover, bacteriophage-based interventions can produce longer-lasting effects by establishing a healthy and diverse gut microbiome. In contrast, many existing antibiotic therapies decrease diversity in the gut microbiome and perturb the gut microbial community, leading to dysbiosis.

Conventional bacteriophage therapy involves the use of bacteriophages that infect a specific bacterial pathogen responsible for an infection. The typical strategy for bacteriophage therapy involves cultivating a single bacteriophage, or a “cocktail” of multiple bacteriophages, that show in vitro efficacy against a specific disease-causing pathogen that was collected from a patient at the site of infection and cultured. Since each bacteriophage has a limited range of susceptible hosts, this strategy relies on careful selection of bacteriophages that are capable of infecting and lysing the bacterial pathogen of interest. If the bacterial pathogen develops resistance to infection by the bacteriophage, this “one bacteriophage against one bacterium” approach can result in a loss of clinical efficacy.

In contrast to the established use of bacteriophage, this disclosure describes an alternative approach of using an entire community of bacteriophages to treat a perturbed microbiome or any dysbiotic condition associated with reduced or loss of bacterial diversity. The approach described herein addresses community-level problems using a community-level solution (a community of bacteriophages). In one or more embodiments, the bacteriophage community may be isolated from a source that includes fermented plant material. Exemplary fermented plant materials include, but are not limited to, appam, atchara, bagoong, balao-balao, bánh cu\hat{ó}n, brem, burong isda, cheonggukjang, cincalok, curtido, dhokla, doenjang, doubanjiang, douzhi, fermented coconut water, fermented pineapple juice, fermented tofu, fermented bean paste, fish sauce, galapong, ganjang, gochujang, gundruk, hákarl, jeotgal, kenkey, khanom chin, kimchi, kombucha, kusaya, lufu, miso, mixian, mohnyin tjin, murri, nata de coco, nata de piña, naem, nattō, nem chua, ngapi, ogi, ogiri, oncom, palappam, peuyeum, pickles, pickle brine, poi, pon ye gyi, sauerkraut, shiokara, shrimp paste, sinki, soweans, soy sauce, sumbala, tarhana, tempeh, tianmianjiang, tungtoh, and Worcestershire sauce.

In one or more embodiments, a bacteriophage preparation may be prepared by isolating the bacteriophages from the source. In other cases, a bacteriophage preparation may be prepared by a method that includes propagating a bacteriophage community in a suitable bacterial culture (e.g., using a bioreactor) and then isolated from the propagational bacterial culture.

The bacteriophage preparation can include members of the family Myoviridae, the family Podoviridae, the family Siphoviridae, the family Inoviridae, the family Microviridae, the family Corticoviridae, the family Tectiviridae, the family Leviviridae, the family Cystoviridae, the family Rudiviridae, the family Fuselloviridae, the family Lipothrixviridae, or the family Plasmaviridae. In preferred embodiments, the bacteriophage preparation includes members of more than one family.

In one or more embodiments, the bacteriophage preparation can be enriched to increase the concentration and/or bioactivity of the bacteriophages. Exemplary methods for enriching the bacteriophage preparation include, but are not limited to, methods that induce bacteriophages to be released from their bacterial host using processes such as UV treatment, mitomycin C, temperature change, high salinity, high basicity, high acidity, or other bacteriophage inducing agents. In other embodiments, the bacteriophage preparation may be enriched by centrifugation, ultracentrifugation, filtration, size fractionation, affinity purification, affinity chromatography, a CsCl density gradient, polyethylene glycol treatment, chloroform treatment, or entrapment of bacterial cells into polymers. In one or more embodiments, cyclical harvesting can be applied to extraction of bacteriophage. Cyclical harvesting is novel in the field of phage biology and involves using selective extraction techniques to harvest specific bacteriophage types from the microbial community at their peak population density. Additionally, the concentration of bacteriophage in a sample could be enriched by adding bacteriophages collected from one or more other sources.

Thus, this disclosure describes a novel therapy for treating gut dysbioses (e.g., SIBO or intestinal bacterial overgrowth) using a treatment that includes bacteriophages that are not specifically matched to a target pathogen and/or a diverse community of bacteriophages. A gut dysbiosis-targeting bacteriophage community may be collected, mixed, and/or separated from a variety of sources including, but not limited to, environmental samples or a fermentation process (e.g., fermented plant material, fermented plant juice, fermented food source, fermented biofuel, fermented bio-waste, fermented feedstock etc.), which is then further cultivated, propagated, and/or enriched in a bacterial culture bioreactor that represents a mixed bacterial and/or bacteriophage community. Administering a diverse population of bacteriophages to the gut microbiome can reduce the density of entire bacterial community, thereby correcting bacterial overgrowth. This community-level approach can also increase overall bacterial diversity in the microbiome, thereby potentially remediating numerous dysbioses, restoring homeostasis in host-gut microbial relationship, and/or treating disorders associated with a decreased bacterial diversity, a shift in bacterial community structure, or an abnormal bloom of pathobionts.

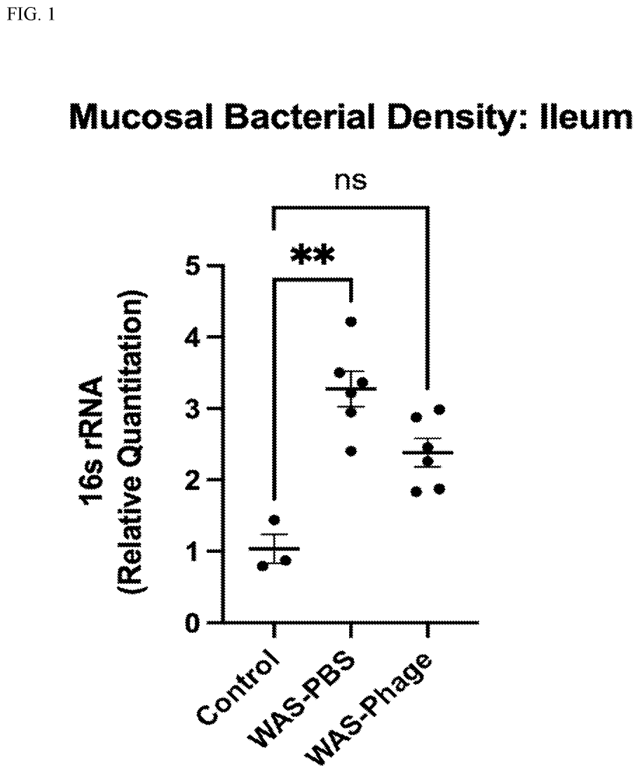

In an exemplary embodiment, a diverse, heterogeneous bacteriophage community was tested as a treatment in a mouse model of dysbiosis, which was induced by water-avoidance stress. The dysbiosis was characterized by bacterial overgrowth in the small intestine (i.e., SIBO). The water-avoidance stress challenge induced dysbiosis in placebo-treated mice (FIG. 1, WAS-PBS) as demonstrated by increased bacterial density. Specifically, the ileum of the WAS-PBS group had a 3.3-fold increase in bacterial density (3.3±0.6) relative to the control group (1.0±0.3; p<0.005). The group that received treatment with the bacteriophage community (WAS-Phage) had a lower bacterial density than the WAS-PBS group. The WAS-Phage group had a bacterial density in the ileum (2.4±0.5) that was not significantly different from the Control group (n.s.).

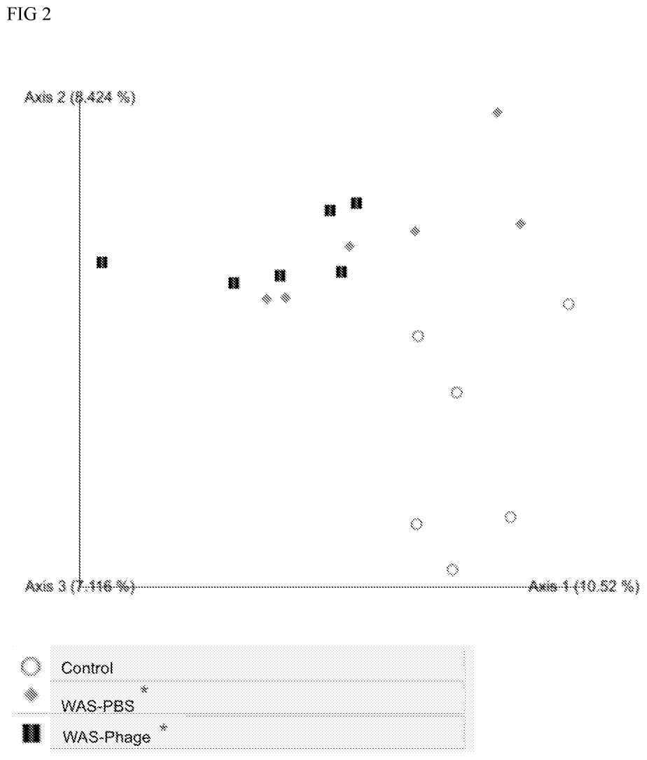

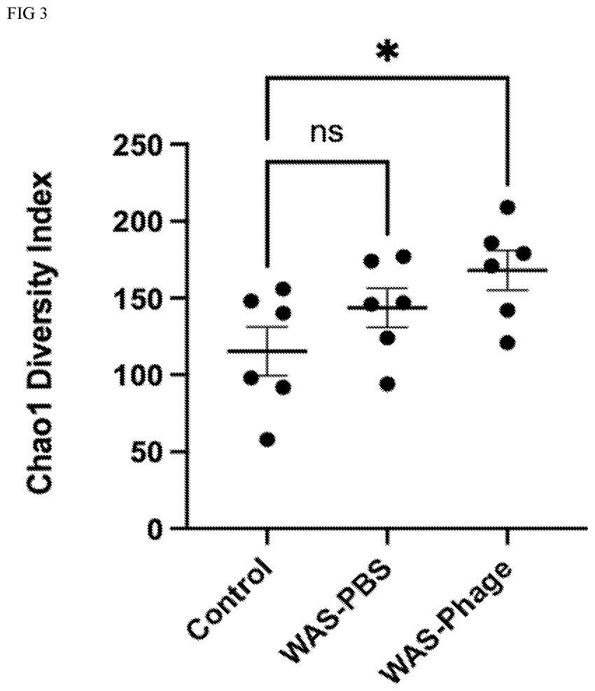

Additionally, sequencing analysis of the cecal microbiome revealed that the WAS-Phage group had a 1.5-fold increase in alpha diversity (mean=168±13) relative to control (mean=115±16; p<0.05) as measured by the Chao1 index (FIG. 3). The Chao1 index of the WAS-PBS group (mean=144±13) was not significantly different from that of the control (n.s.). These statistics were determined using the Kruskal-Wallis test with Dunn's correction for multiple comparisons. Analysis of the beta diversity between experimental groups demonstrated that the WAS-testing was sufficient for changing microbiome community composition. Both WAS groups, WAS-PBS and WAS-Phage, were significantly different from the Control group (p<0.01), using Bray-Curtis dissimilarity (FIG. 2). The beta diversity data, interpreted with the alpha diversity analysis, demonstrate that WAS-testing altered the overall microbiome community composition, relative to control, which demonstrates dysbiosis. Further, treatment with the bacteriophage preparation was the only treatment associated with an overall increase in diversity of species present in the microbiome, an indication that it may have utility in remediating dysbiosis.

These findings provide evidence that in a subject with experimentally induced gut microbiome dysbiosis, treatment with a bacteriophage community is effective at returning bacterial density in the ileum to the level of healthy, untreated control. These data on the resolution of SIBO in mice is the first evidence of the use of a diverse, heterogeneous bacteriophage community that was cultured in an artificial environment using a source that includes fermented plant material, as a treatment for gut microbial dysbiosis.

Thus, this disclosure describes bacteriophage preparations and the use of bacteriophage preparations for the treatment of conditions caused by dysbiosis. As used herein, “treat” or variations thereof refer to reducing, limiting progression, ameliorating, or resolving, to any extent, the symptoms or signs related to a condition. A “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Generally, a “therapeutic” treatment is initiated after the condition manifests in a subject, while “prophylactic” treatment is initiated before a condition manifests in a subject.

Treatment that is prophylactic—e.g., initiated before a subject manifests a symptom or clinical sign of the condition such as, for example, while an infection remains subclinical—is referred to herein as treatment of a subject that is “at risk” of having the condition. As used herein, the term “at risk” refers to a subject that may or may not actually possess the described risk. Thus, for example, a subject “at risk” of infectious condition is a subject present in an area where other individuals have been identified as having the infectious condition and/or is likely to be exposed to the infectious agent even if the subject has not yet manifested any detectable indication of infection by the microbe and regardless of whether the subject may harbor a subclinical amount of the microbe. As another example, a subject “at risk” of a non-infectious condition is a subject possessing one or more risk factors associated with the condition such as, for example, genetic predisposition, ancestry, age, sex, geographical location, lifestyle, or medical history. In addition, a subject may be considered “at risk” because of exposure or anticipated exposure to a factor that perturbs the gut microbiome such as a course of antibiotics, an episode of acute gastroenteritis, an episode of surgery or general anesthesia, prolonged stress, deployment to a combat zone, travel, illness, etc. As yet another example, a subject who had been treated for dysbiosis is “at risk” for a recurrence or relapse of dysbiosis. Prophylactic treatment may be initiated before a subject manifests symptoms or clinical signs of the condition or when a subject manifests prodromal symptoms or clinical signs that are harbingers of a full-blown recurrence or relapse of dysbiosis. Such prophylactic treatment may be directed at reducing the likelihood and/or severity of any recurrence or relapse or reduce of dysbiosis. For example, a fluctuating energy level may be a prodromal symptom of an impending recurrence or relapse of dysbiosis in chronic fatigue syndrome. As another example, a subject with idiopathic REM sleep behavior disorder (iRBD), a prodromal condition that progresses to Parkinson's disease, could be treated before the condition evolves to full blown Parkinson's disease.

Accordingly, a bacteriophage preparation may be administered to a subject before, during, or after the subject first exhibits a symptom or clinical sign of the condition caused by dysbiosis. Treatment initiated before the subject first exhibits a symptom or clinical sign associated with the condition may result in decreasing the likelihood that the subject experiences clinical evidence of the condition compared to a subject to which the bacteriophage preparation is not administered, decreasing the severity of symptoms and/or clinical signs of the condition, and/or completely resolving the condition. Treatment initiated after the subject first exhibits a symptom or clinical sign associated with the condition may result in decreasing the severity of symptoms and/or clinical signs of the condition compared to a subject to which the bacteriophage preparation is not administered, and/or completely resolving the condition.

Thus, the method includes administering an effective amount of the composition to a subject having, or at risk of having, a condition caused by dysbiosis. In this aspect, an “effective amount” is an amount effective to reduce, limit progression, ameliorate, or resolve, to any extent, a symptom or clinical sign related to the condition.

The bacteriophage preparation described herein may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic bacteriophage preparations is contemplated. Supplementary active ingredients also can be incorporated into the bacteriophage preparations. As used herein, “pharmaceutically acceptable” refers to a material that is not biologically or otherwise undesirable, i.e., the material may be administered to an individual along with bacteriophage preparation without causing any undesirable biological effects or interacting in a deleterious manner with any of the other components of the pharmaceutical bacteriophage preparation in which it is contained.

The bacteriophage preparation may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), via a tube placed in an organ such as the gastrointestinal tract or a body cavity, or topical (e.g., intranasal, intrapulmonary, intramammary, intravaginal, intrauterine, intradermal, transcutaneous, rectally, intracystic, etc.). A pharmaceutical composition can be administered to a mucosal surface, such as by administration to, for example, the nasal mucosa, respiratory mucosa, vaginal mucosa, rectal mucosa, etc. (e.g., by spray or aerosol) or applied to the skin or scalp (e.g., cream, gel, solution, ointment, spray, or patch). A composition also can be administered via a sustained or delayed release.

Thus, the bacteriophage preparation may be provided in any suitable form including but not limited to a solution, a suspension, an emulsion, a spray, an aerosol, or any form of mixture. The bacteriophage preparation may be delivered in formulation with any pharmaceutically acceptable excipient, carrier, or vehicle. For example, the formulation may be delivered in a conventional topical dosage form such as, for example, a cream, an ointment, a skin patch, an aerosol formulation, a non-aerosol spray, a gel, a lotion, a hand wash, a body wash, a shampoo, a surgical or dental wash or rinse, a dentrifice, an eye drop, an inhaler, and the like.

Formulations designed for oral delivery include, but are not limited to, microspheres (coated, uncoated, or a combination of coated and uncoated), particles (coated, uncoated, or a combination of coated and uncoated), a powder, a granule formulation, a suspension, an emulsion, a solution, a syrup, an elixir, tablets (coated, uncoated, or a combination of coated and uncoated), a troche, a capsule, a caplet, a lozenge, a dentrifice, a chewing gum, or a spray.

Formulations designed for delivery to skin include, but are not limited to, microspheres (coated, uncoated, or a combination of coated and uncoated), particles (coated, uncoated, or a combination of coated and uncoated), a powder, a granule formulation, a suspension, an emulsion, a solution, a hand or body wash, a shampoo, a patch, or a spray.

Formulations designed for delivery to the eye or conjunctiva include, but are not limited to, microspheres (coated, uncoated, or a combination of coated and uncoated), particles (coated, uncoated, or a combination of coated and uncoated), a powder, a granule formulation, a suspension, an emulsion, or a solution, and may be delivered as an eye drop or as a spray.

Formulations designed for delivery to the nasal or respiratory tract include, but are not limited to, microspheres (coated, uncoated, or a combination of coated and uncoated), particles (coated, uncoated, or a combination of coated and uncoated), a powder, a granule formulation, a suspension, an emulsion, or a solution, and may be delivered as an aerosol or a spray.

The formulation may further include one or more additives including such as, for example, an adjuvant, a skin penetration enhancer, a colorant, a fragrance, a flavoring, a moisturizer, a thickener, and the like.

In some cases, the method of treatment may involve treating dysbiosis localized to an epithelial tissue such as, for example, the epidermis, a mucosal surface, at least a portion of the skin or scalp, at least a portion of the oral cavity including tooth or gum, at least a portion of the gastrointestinal tract, at least a portion of the nasal cavity, at least a portion of the respiratory tract, at least a portion of the genito-urinary tract, or a body cavity. Formulations designed for delivery to the genito-urinary tract (e.g., the vagina or rectum) include, but are not limited to, microspheres (coated, uncoated, or a combination of coated and uncoated), particles (coated, uncoated, or a combination of coated and uncoated), a powder, a granule formulation, a suspension, an emulsion, a solution, a syrup, an elixir, tablets (coated, uncoated, or a combination of coated and uncoated), a troche, a douche, an enema, a capsule, a caplet, a lozenge, a suppository, or a spray. Accordingly, the bacteriophage preparation may be provided in a formulation suitable for delivery to an epithelial tissue by a route of administration suitable for delivery to the epithelial tissue.

In one or more embodiments, the bacteriophage preparation is incorporated into a medical device such as, for example, a pacemaker, a catheter, or a stent. As used herein, “incorporated into a medical device” refers to any manner of associating the bacteriophage preparation with the medical device. Exemplary ways in which the bacteriophage preparation may be incorporated into a medical device include, but are not limited to, the bacteriophage preparation being integrated into one or more components of the medical device, the bacteriophage preparation being included in a coating applied to a portion of the medical device, or the bacteriophage preparation being included in a patch or reservoir of the medical device.

A formulation may be conveniently presented in unit dosage form and may be prepared by methods well known in the art of pharmacy. Methods of preparing a pharmaceutical composition with a pharmaceutically acceptable carrier include the step of bringing the bacteriophage preparation into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

A dose of the bacteriophage preparation administered can vary depending on various factors including, but not limited to, the weight, physical condition, and/or age of the subject, and/or the route of administration. Thus, the concentration of bacteriophage in a given volume of the formulation can vary widely, and depends upon factors such as the species, age, weight, and physical condition of the subject, and/or the method of administration. Accordingly, it is not practical to set forth generally the amount that constitutes an amount of the bacteriophage preparation effective for all possible applications. Those of ordinary skill in the art, however, can readily determine the appropriate amount with due consideration of such factors.

In one or more embodiments, the method can include administering sufficient amount of a bacteriophage preparation to provide a dose of, for example, from about 10⁴ bacteriophages/ml to about 10¹² bacteriophages/ml to the subject, although in one or more embodiments the methods may be performed by administering the bacteriophage preparation in a dose outside this range. In some of these embodiments, the method includes administering sufficient bacteriophage preparation to provide a dose of from 10⁵ bacteriophages/ml to about 10¹⁰ bacteriophages/ml to the subject, from about 4×10¹⁰ bacteriophages/ml to about 3×10¹¹ bacteriophages/ml to the subject, for example, or a dose of from about 5×10¹⁰ bacteriophages/ml to about 1.2×10¹¹ bacteriophages/ml to the subject. In one exemplary embodiment, a dose of about 10⁷ bacteriophages/ml are administered to the subject.

A single dose may be administered all at once, continuously for a prescribed period of time, or in multiple discrete administrations. When multiple administrations are used, the amount of each administration may be the same or different. For example, a dose of 10⁷ bacteriophages per day may be administered as a single administration of 10⁷ bacteriophages, continuously over 24 hours, as two administrations of 5×10⁶ bacteriophages, or as unequal administrations in a 24-hour period that total 10⁷ bacteriophages. When multiple administrations are used to deliver a single dose, the interval between administrations may be the same or different.

In certain embodiments, the bacteriophage preparation may be administered as a single one-off dose to multiple doses per week, although in one or more embodiments the method can involve a course of treatment that includes administering doses of the bacteriophage at a frequency outside this range. When a course of treatment involves administering multiple doses within a certain period, the amount of each dose may be the same or different. For example, a course of treatment can include a loading dose initial dose, followed by a maintenance dose that is lower than the loading dose. Also, when multiple doses are used within a certain period, the interval between doses may be the same or be different.

In one or more embodiments, the bacteriophage preparation may be administered after a sustained period of nutrient deprivation. As used herein, a “sustained period of nutrient deprivation” refers to a time longer than the usual time between meals. As used herein, “nutrient deprivation” refers to any reduced access to calories or nutrients. Thus, in one or more embodiments, a sustained period of nutrient deprivation can include fasting for a minimum period of at least eight hours such as, for example, at least 12 hours, and least 18 hours, at least 24 hours, at least 30 hours, or at least 36 hours. A sustained period of nutrient deprivation can include fasting for a maximum period of no more than 72 hours such as, for example, no more than 60 hours, no more the 54 hours, no more than 48 hours, no more than 42 hours, no more than 36 hours, no more than 30 hours, no more than 24 hours, or no more than 18 hours. In some cases, a sustained period of nutrient deprivation can include fasting for a period within a range having endpoints defined by any minimum fasting period set forth above and any maximum fasting period set forth above that is greater than the minimum fasting period. In certain embodiments, the sustained period of nutrient deprivation can include fasting for 12 hours to 36 hours. in one specific exemplary embodiment, the sustained period of nutrient deprivation can include fasting for 24 hours.

Nutrient deprivation reduces available food sources for resident gut bacteria, thus inhibiting the growth of gut bacterial populations. When used in conjunction with bacteriophage treatment, nutrient deprivation can weaken resident gut bacteria thereby making administration of the bacteriophage treatment more effective. In one or more alternative embodiments, bacteriophage treatment may be administered prior to, during, or after one or more meals.

In one or more embodiments, the bacteriophage preparation may be administered, for example, for a period ranging from a single dose to the remaining lifespan of the subject. In certain embodiments, the bacteriophage preparation may be administered as a single one-off dose. In other embodiments, the bacteriophage preparation may be administered for a period of one day to a period of about one year, such as, for example, for a period of from about three days to about seven days. Treatment with bacteriophage preparation also may occur periodically as a retreatment to reduce the likelihood and/or severity of a recurrence of dysbiosis.

In one or more embodiments, the method can include administering to the subject a second pharmaceutical composition for treating dysbiosis. Exemplary pharmaceutical compositions for treating dysbiosis include, but are not limited to, an antibiotic, a prebiotic, a probiotic, a synbiotic, a fecal microbiota transplant, membrane vesicles, an autophagy inducer, or a combination of any two or more pharmaceutical compositions for treating dysbiosis.

Exemplary antibiotics include, but are not limited to, rifaximin, metronidazole, cephalexin, trimethoprim-sulfomethoxazole, doxycycline, colistin, tetracycline, clindamycin, ciprofloxacin, levofloxacin, moxifloxacin, ofloxacin, norfloxacin, ampicillin, amoxicillin erythromycin, tinidazole, nitazoxanide, albendazole, paromomycin, quinacrine, lactulose, bismuth subsalicylate, gentamicin, neomycin, or any combination of two or more antibiotics. Exemplary prebiotics include, but are not limited to, fructo-oligosaccharides, disaccharides, monosaccharides, polyols, galacto-oligosaccharides, inulin, short chain carbohydrates, sugar alcohols, oligofructose, or a combination of two or more prebiotics.

Exemplary probiotics include, but are not limited to, a member of the Lactic acid bacteria group such as, for example, a Pediococcus, a Streptococcus, a Lactococcus, a Lactobacillus, an Oenococcus, a Weissella, or a Leuconostoc spp. such as Lactobacillus acidophilus, Lactobacillus plantarum, Lactobacillus casei, Lactobacillus paracasei, Lactobacillus delbrueckii, Lactobacillus brevis, Lactobacillus fermentum, Lactobacillus johnsonii, Lactobacillus delbrueckii, Lactobacillus salivarius, Lactobacillus sakei, Lactobacillus bulgarius, Lactobacillus reuteri, Lactobacillus rhamnosus GG, Lactobacterium lactis, Streptococcus thermophilus, Saccharomyces boulardii, Saccharomyces bayanus, Saccharomyces cerevisiae, Bifidobacterium bifidum, Bifidobacterium infantis, Bifidobacterium adolescentis, Bifidobacterium longum, Bifidobacterium breve, Bifidobacterium lactis, Bifidobacterium animalis, Bacillus coagulans, Bacillus subtilis, Bacillus cereus, Pedicoccus acdilactici, Leuconostoc mesenteroides, Escherichia coli Nissle, Enterococcus durans, Weissella cibaria, Enterococcus faecium, or a combination of any two or more probiotics.

Exemplary synbiotic agents include, but are not limited to, a combination of a prebiotic and a probiotic.

Exemplary fecal microbiota transplants include, but are not limited to, whole fecal material, partial fecal material, altered fecal material, whole microbiota, partial microbiota, fecal filtrate, or altered microbiota.

In one or more embodiments, the dysbiosis being treated can include dysbiosis localized to an epithelial tissue such as, for example, the epidermis, a mucosal surface, at least a portion of the skin or scalp, at least a portion of the oral cavity including tooth or gum, at least a portion of the gastrointestinal tract, at least a portion of the nasal cavity, at least a portion of the respiratory tract, at least a portion of the genito-urinary tract, or a body cavity.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Reference throughout this specification to “one embodiment,” “an embodiment,” “certain embodiments,” or “one or more embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be conducted in any feasible order. And, as appropriate, any combination of two or more steps may be conducted simultaneously.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

The present invention is illustrated by the following examples. It is to be understood that the particular examples, materials, amounts, and procedures are to be interpreted broadly in accordance with the scope and spirit of the invention as set forth herein.

### EXAMPLES

Preparations of diverse and heterogeneous communities of bacteriophages were produced in a spontaneous lactic acid bacteria culture of raw cabbage in 3% NaCl w/v solution. Cultures were incubated at 18° C. for 21 days and then were subjected to centrifugation, filtration, and concentration to isolate a pure preparation of bacteriophages. Centrifugation of the sample at 5000×g for 30 minutes pelleted bacterial cells and particulate matter from the culture. The remaining supernatant, in which the bacteriophages were contained, was filtered at 450 nm to filter-sterilize the solution and further remove any bacteria that remained. A centrifugal filter with a 3 nm pore size, a pore size known to be small enough to capture all known bacteriophages, was used to concentrate and isolate the bacteriophages. Bacteriophages were concentrated to 10× the level of the original culture by subsequent filling and centrifugation of the centrifugal filter. The bacteriophages were cleaned on the centrifugal filter using phosphate-buffered saline (PBS) solution. Presence of bacteriophages in the solution was confirmed using electron microscopy.

To establish a representative gut dysbiosis model in which to test the bacteriophage preparation, C57BL/6 mice (n=6) were challenged with water-avoidance stress (WAS-PBS) testing to induce SIBO. WAS testing involved 10 days of one-hour treatments in which each mouse was placed on a circular platform (2-inch diameter) in the middle of a clear plastic container that was filled with water up to 1 cm below the surface of the platform. Concurrently, another group received WAS testing as well as the bacteriophage preparation (WAS-Phage; n=6). WAS-Phage mice received one treatment of approximately 10⁷ bacteriophage preparation per day, beginning three days prior to the start of the WAS testing and lasting for until the last day of the WAS testing for a total of 13 treatments. The WAS-PBS group received PBS gavage in place of the bacteriophage preparation. During this period, the Control mice (n=3) remained in their cages. Mice were euthanized 24 hours after the WAS and WAS-Phage groups received their last treatment. Tissue samples were collected from the ileum of the small intestine and stored in Zymo RNA/DNA Shield prior to analysis.

For quantitation of the bacterial density in the ileum of the small intestine, DNA was extracted from approximately 50 mg of ileal tissue by processing using the DNeasy Blood and Tissue Kit (Qiagen). Copies of the gene for the 16s rRNA gene subunit, the universal bacterial marker, were enumerated in the extracted ileal DNA using quantitative polymerase chain reaction (qPCR). By using a primer pair that targets the 16s rRNA gene, one is able to enumerate the amount of bacterial cells present in a sample relative to the amount of eukaryotic cells of the host tissue, which can be identified using the 18s rRNA gene. Using this approach allows for the accurate enumeration of mucosally associated bacteria in different tissues by comparing the quantity of bacterial cells relative to the quantity of eukaryotic host cells, thereby getting a measure for bacterial density in the host tissue. Data from the qPCR were analyzed using the ^{ΔΔ}Ct method and results are reported as fold-change (mean±SE) relative to the Control group which received no treatment. The difference between the means of each group was analyzed using the Kruskal-Wallis Test with Dunn's correction for multiple comparisons. All statistical analyses were performed with PRISM (GraphPad Software, Inc., San Diego, CA).

For comparison of alpha and beta diversity between treatment groups, approximately 200 mg of fecal material was collected from the cecum of each animal and sent to the University of Minnesota Genomics Center (UMGC) for extraction and 16S sequencing of microbial DNA. 16S microbiome sequencing allows for the characterization of the full microbial community composition. Bioinformatic analysis of the 16S sequencing data was performed on a fee basis by CD Genomics (Shirley, NY). This analysis compares microbial community composition between treatment groups, which results in quantitative measurements of both community diversity (i.e., alpha diversity) as well as dissimilarity between groups (i.e., beta diversity). Alpha diversity indices, such as the Chao1 index used in this study, quantify the number of species, resulting in an objective measurement of diversity within a microbial community. Conversely, beta diversity dissimilarity, such as the Bray-Curtis dissimilarity used in this study, is a subjective measurement that compares the differences in microbial community composition between groups; specifically, comparing groups based on which species are present and their abundances.

For statistical analyses, the difference between the alpha diversity means of each group was analyzed using the Kruskal-Wallis Test with Dunn's correction for multiple comparisons. All statistical analyses were performed with PRISM (GraphPad Software, Inc., San Diego, CA). Beta diversity dissimilarities were analyzed using Permanova pairwise comparisons on a fee basis by CD Genomics (Shirly, CA).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A pharmaceutical composition comprising:
a preparation of a heterogeneous bacteriophages obtained from a fermentative process; and
a pharmaceutically-acceptable carrier.

2. The pharmaceutical composition of claim 1, further comprising an adjuvant.

3. The pharmaceutical composition of claim 1, wherein the bacteriophages comprise members of the family Myoviridae, members of the family Podoviridae, members of the family Siphoviridae, members of the family Inoviridae, members of the family Microviridae, members of the family members of the family Corticoviridae, members of the family Tectiviridae, members of the family Leviviridae, members of the family Cystoviridae, members of the family Rudiviridae, members of the family Fuselloviridae, members of the family Lipothrixviridae, members of the family Plasmaviridae, or bacteriophages from a combination of the foregoing families.

4. The pharmaceutical composition of claim 1, wherein the fermentative process produces appam, atchara, bagoong, balao-balao, bánh cu\hat{ń}, brem, burong isda, cheonggukjang, cincalok, curtido, dhokla, doenjang, doubanjiang, douzhi, fermented coconut water, fermented grain, fermented grapes, fermented pineapple juice, fermented tofu, fermented bean paste, fish sauce, galapong, ganjang, gochujang, gundruk, hákarl, jeotgal, kefir, kenkey, khanom chin, kimchi, kombucha, kusaya, lufu, miso, mixian, mohnyin tjin, murri, nata de coco, nata de piña, naem, nattō, nem chua, ngapi, ogi, ogiri, oncom, palappam, peuyeum, pickles, pickle brine, poi, pon ye gyi, sauerkraut, shiokara, shrimp paste, sinki, soweans, soy sauce, sumbala, tarhana, tempeh, tianmianjiang, tungtoh, or Worcestershire sauce.

5. The pharmaceutical composition of claim 1, wherein the fermentative process produces a fertilizer or livestock feed.

6. The pharmaceutical composition of claim 1, wherein the fermentative process comprises fermentation of a plant material, a plant juice, a feedstock, a biofuel, or a bio-waste.

7. The pharmaceutical composition of claim 1, formulated for delivery to epithelial tissue.

8. The pharmaceutical composition of claim 7, wherein the epithelial tissue comprises epidermis, a mucosal surface, at least a portion of the skin or scalp, at least a portion of the oral cavity, at least a portion of the gastrointestinal tract, at least a portion of the nasal cavity, at least a portion of the respiratory tract, at least a portion of the genito-urinary tract, at least a portion of the conjunctiva, or a body cavity.

9. The pharmaceutical composition of claim 7, wherein the pharmaceutical composition comprises the bacteriophage preparation formulated into a microsphere, a particle, a powder, a granule, a suspension, an emulsion, a solution, an elixir, a syrup, a tablet, a troche, a douche, a suppository, a capsule, a lozenge, a gum, a spray, a body wash, a shampoo, a patch, a strip, a dentrifice, an eye drop, or an aerosol.

10. A method of preparing a bacteriophage preparation, the method comprising:
isolating bacteriophage from a fermentative process; and
combining the isolated bacteriophage with a pharmaceutically acceptable carrier.

11. The method of claim 10, further comprising enriching the bacteriophage preparation, wherein enriching the bacteriophage preparation:
increases the concentration of bacteriophages in the preparation; or
increases the bioactivity of bacteriophages in the preparation.

12. (canceled)

13. The method of claim 11, wherein the bacteriophage preparation is enriched by treating the bacteriophage preparation with UV light, mitomycin C, a temperature change, increased salinity, a change in pH, or another bacteriophage inducing treatment.

14. The method of claim 10, further comprising culturing the bacteriophage preparation with suitable bacterial species in a bioreactor.

15. The method of claim 10, wherein the fermentative process produces appam, atchara, bagoong, balao-balao, bánh cu\hat{ó}n, brem, burong isda, cheonggukjang, cincalok, curtido, dhokla, doenjang, doubanjiang, douzhi, fermented coconut water, fermented grain, fermented grapes, fermented pineapple juice, fermented tofu, fermented bean paste, fish sauce, galapong, ganjang, gochujang, gundruk, hákarl, jeotgal, kenkey, khanom chin, kefir, kimchi, kombucha, kusaya, lufu, miso, mixian, mohnyin tjin, murri, nata de coco, nata de piña, naem, nattō, nem chua, ngapi, ogi, ogiri, oncom, palappam, peuyeum, pickles, pickle brine, poi, pon ye gyi, sauerkraut, shiokara, shrimp paste, sinki, soweans, soy sauce, sumbala, tarhana, tempeh, tianmianjiang, tungtoh, or Worcestershire sauce.

16. The method of claim 10, wherein the fermentative process produces a fertilizer or livestock feed.

17. The method of claim 10, wherein the fermentative process comprises fermentation of a plant material, a plant juice, a feedstock, a biofuel, or a bio-waste.

18. A method of treating dysbiosis in a subject having, or at risk of having, dysbiosis, the method comprising:
administering to the subject a bacteriophage preparation in an amount effective to ameliorate at least one symptom or clinical sign of dysbiosis.

19. The method of claim 18, wherein the method further comprises administering to the subject a second pharmaceutical composition for treating dysbiosis.

20. The method of claim 19, wherein the second pharmaceutical composition for treating dysbiosis comprises an antibiotic, a prebiotic, a probiotic, a synbiotic agent, a fecal microbiota transplant, membrane vesicles, or an autophagy inducer.

21-27. (canceled)

28. The method of claim 18, wherein the dysbiosis is localized to an epithelial tissue.

29. (canceled)

30. The method of claim 18, wherein the bacteriophage preparation is incorporated into a medical device.

31. (canceled)

32. The method of claim 18, wherein the dysbiosis causes bacterial overgrowth.

33. The method of claim 18, further comprising subjecting the subject to a sustained period of nutrient deprivation prior to administering the bacteriophage preparation to the subject.

34. The method of claim 33, wherein the sustained period of nutrient deprivation is about one to three days.

35. The method of claim 18, wherein the bacteriophage preparation is administered before, during and after one or more meals.

36. The method of claim 18, wherein the bacteriophage preparation is administered orally, administered topically, administered through inhalation, or administered parenterally.

37. A method of preparing or priming the gut environment of a healthy subject for administering a source of bacteria, the method comprising:
administering to the subject a bacteriophage preparation in an amount effective to improve receptivity of subject's gut to the source of bacteria.

38. The method of claim 37, wherein the source of bacteria comprises a probiotic or a fecal microbiota transplant.

39. The method of claim 37, wherein the bacteriophage preparation is administered by mouth, delivered directly to the gut, or encapsulated as a microbial transplant.

40. The method of claim 37, further comprising subjecting the subject to a sustained period of nutrient deprivation prior to administering the bacteriophage preparation to the subject.

41. The method of claim 37, wherein the sustained period of nutrient deprivation is about one to three days.

42. The method of claim 37, wherein the bacteriophage preparation is administered before, during and after one or more meals.
